(19) United States
(12) Patent Application Publication
(10) Pub. No.: US 2015/0104445 A1
(43) Pub. Date: Apr. 16, 2015

(54) METHODS OF INHIBITING THE ALTERNATIVE PATHWAY OF COMPLEMENT IMMUNE SYSTEM ACTIVATION AND COMPOSITIONS USED THEREIN

(71) Applicant: ViroPharma Holdings Limited, Hamilton (BM)

(72) Inventors: Marc E. Uknis, Chadds Ford, PA (US); Jo Anne M. Saye, Hockessin, DE (US); Colin Broom, Devon, PA (US)

(21) Appl. No.: 14/511,314

(22) Filed: Oct. 10, 2014

Related U.S. Application Data
(60) Provisional application No. 61/889,175, filed on Oct. 10, 2013.

(51) Int. Cl. A61K 38/57; A61K 38/17; A61K 39/395

(52) U.S. Cl. CPC A61K 38/57; A61K 39/3955; A61K 38/17; A61K 2039/505
USPC 424/133.1; 514/20.3; 514/1.4; 514/13.5

(57) ABSTRACT

Methods and compositions are provided for treating diseases implicating alternative pathway complement immune system activation.

### CROSS-REFERENCE TO RELATED APPLICATIONS

The present application claims the benefit of U.S. Provisional Patent Application No. 61/889,175, filed Oct. 10, 2013, the entire disclosure of which is incorporated by reference herein.

### FIELD OF THE INVENTION

The present invention relates generally to methods and compositions for treating diseases implicating alternative pathway complement immune system activation and more particularly, but not exclusively, to methods of treating diseases such as paroxysmal nocturnal hemoglobinuria (PNH) by administering a therapeutically effective amount of a C1-esterase inhibitor (C1-INH).

### BACKGROUND OF THE INVENTION

Several diseases are known to implicate alternative pathway complement (APC) system activation. For example, paroxysmal nocturnal hemoglobinuria (PNH) is a rare, clonal, hematopoietic stem cell disorder that manifests with a complement-mediated hemolytic anemia, bone marrow failure, and a propensity for thrombosis. PNH is life threatening and may develop independently (i.e., primary PNH) or as a consequence of other disorders (i.e., secondary PNH). PNH patients experience hemolysis in the context of underlying complement activation. Hemolysis is characterized by the abnormal breakdown of red blood cells, either in the blood vessels (i.e., intra-vascular hemolysis) or elsewhere in the human body (i.e., extravascular hemolysis). Furthermore, PNH is accompanied by a deficiency of glycosylphosphatidylinositol (GPI) anchored proteins that protect red blood cells at different stages of the complement cascade. Specifically, PNH results in deficiencies of both CD55 and CD59. CD55 regulates the formation and stability of the C3 and C5 convertases, whereas CD59 blocks the formation of the membrane attack complex (MAC). One of the few treatments available for PNH includes eculizumab (Soliris®; see, for example, U.S. Pat. Nos. 6,074,642 and 6,355,245; and U.S. Patent Application Publication No. 2012/0237515, each of the foregoing patents and patent publications are incorporated by reference herein).

Chronic treatment with eculizumab to block C5 and subsequent formation of the end product of complement activation, the membrane attack complex (MAC), results in sustained control of intravascular hemolysis, leading to transfusion independence for those who are complete responders to the therapy. Eculizumab operates by compensating for the CD59 deficiency on PNH red blood cells. However, eculizumab has not been observed to compensate for the CD55 deficiency. In patients receiving eculizumab, PNH red blood cells begin to accumulate C3 on their surface, which leads to opsonization resulting in extravascular hemolysis, as well as ongoing intravascular (direct) hemolysis via APC activation. Thus, the treatment of some PNH patients with eculizumab is incomplete and in others ineffective.

Consequently, there is an unmet clinical need for a complement inhibitor, which exhibits, for example, activity early in the complement cascade, and which would not only reduce C3 accumulation on PNH red blood cells, but also inhibit C3 activation by the alternative complement pathway. The present invention meets that need.

### SUMMARY OF THE INVENTION

The present invention relates to methods and compositions for treating or delaying the progression of diseases or disorders that implicate alternative pathway complement immune system activation and/or the accumulation of protein degradation products of a protein on red blood cells.

In a first aspect, the present invention includes a method of treating or delaying the progression of a disorder alleviated by inhibiting alternative pathway complement immune system activation in a patient in need of such treatment, the method comprising administering a therapeutically effective amount of C1-esterase inhibitor (C1-INH). The C1-INH may comprise a human plasma-derived C1-INH (hC1-INH) or a recombinant C1-INH (rC1-INH). Moreover, the disorder may be selected from the group consisting of paroxysmal nocturnal hemoglobinuria (PNH), dense deposit disease (DDD), factor H deficiency, age-related macular degeneration (AMD), atypical hemolytic uremic syndrome (aHUS), and sepsis. Additionally, the method of the invention may comprise administering an additional biologically active agent effective for treating or delaying the progression of a disorder selected from the group consisting of PNH, DDD, factor H deficiency, AMD, aHUS, and sepsis. The method may comprise, for example, administering eculizumab, TT30, or a combination thereof, as the additional biologically active agent.

In another aspect, the invention includes a method of treating or delaying the progression of a disorder alleviated by inhibiting the accumulation of protein degradation products of a protein on red blood cells in a patient in need of such treatment, the method comprising administering a therapeutically effective amount of C1-esterase inhibitor (C1-INH).

Regarding other aspects, the invention also includes a method of treating or delaying the progression of PNH in a patient in need of such treatment, the method comprising administering therapeutically effect amounts of at least a C1-esterase inhibitor (C1-INH) and eculizumab. In one embodiment, the C1-INH and a C5 inhibitor (e.g., eculizumab) may be administered concurrently or sequentially.

In a further aspect, the invention provides a method of treating the opsonization of blood cells in an organ in a patient in need of such treatment, the method comprising administering a therapeutically effective amount of a C1-esterase inhibitor (C1-INH). In one embodiment, the C1-INH may comprise, for example, a human plasma derived C1-INH (hC1-INH) or a recombinant C1-INH (rC1-INH). In another embodiment, the organ may be selected from the group consisting of spleen, liver, and a combination thereof. In a preferred embodiment, the opsonization of blood cells is due to eculizumab treatment.

In an additional aspect, the present invention includes a pharmaceutical composition for treating or delaying the progression of a disorder alleviated by inhibiting alternative pathway complement immune system activation in a patient in need of such treatment. The composition may comprise a C1-esterase inhibitor (C1-INH); a biologically active agent selected from the group consisting of eculizumab, TT30, or a combination thereof; and a pharmaceutically acceptable carrier medium.

### BRIEF DESCRIPTION OF THE DRAWINGS

The foregoing summary and the following detailed description of the exemplary embodiments of the present invention may be further understood when read in conjunction with the appended drawings, in which:

FIG. 1 schematically illustrates the complement cascade wherein C1 esterase inhibitor (C1-INH) acts in the classical and alternative pathways.

FIG. 2 is a table setting forth patient clinical data in five PNH patients subjected to eculizumab therapy. The patients were annotated by numbers for the first experiments and letters for the second experiments. All data were measured in validated assays.

FIG. 3 is a cell counting analysis that graphically illustrates CD55 expression on erythrocytes of PNH patients treated with eculizumab. Cell counts were obtained in five patients as demonstrated in five graphs. Patients 1-3 have type III PNH cells while patients 4-5 have type II PNH cells. In each of the cell count histograms (A) represents the immunohistological control; (B) represents the PNH cells; and (C) represents a control.

FIG. 4 is an inhibition study that graphically illustrates the concentration dependence of lysis inhibition in PNH erythrocytes by C1-INH. Results shown are the mean absorbance±standard deviation (n=3). In the graph, (A) represents acidified normal serum; and (B) represents acidified heat-inactivated serum. Acidification activates the alternative pathway of complement.

FIG. 5 is a hemolysis study that graphically illustrates the association between lysis and PNH erythrocyte clone size ex vivo. In the graph, (A) represents Patient 1 having Type III PNH cells (clone size 95%); (B) represents Patient 2 having Type III PNH cells (clone size 90%); (C) represents Patient 3 having Type II PNH cells (clone size 65%); and (D) represents Patient 4 having Type II PNH cells (clone size 25%).

FIG. 6 is a hemolysis study that graphically demonstrates that erythrocytes from healthy donors do not lyse. In the graph, lines A-C represent individual healthy patients.

FIG. 7 is a flow cytometry study that graphically illustrates the deposition of C3 on erythrocytes in PNH patients.

FIG. 8 is a flow cytometry study that graphically illustrates the deposition of C3 on erythrocytes in healthy donors.

### DETAILED DESCRIPTION OF THE INVENTION

Paroxysmal nocturnal hemoglobinuria (PNH) is a rare, clonal, hematopoietic stem cell disorder that triggers complement-mediated hemolytic anemia. Somatic mutations in PIG-A, a gene whose product is required for the synthesis of glycosyl-phosphatidylinositol (GPI) anchors, are found in virtually all PNH patients. PNH is marked by a deficiency of GPI anchored proteins in red blood cells at different stages of the complement cascade. GPI anchors tether dozens of proteins to cellular membranes. Thus, PNH cells are deficient in all GPI anchored proteins including the complement regulatory proteins CD55 and CD59. CD55 protein regulates the formation and stability of C3 and C5 convertase. Specifically, CD55 regulates the formation of the membrane attack complex (MAC), an end product of the complement cascade. However, the chronic hemolytic anemia in PNH is largely mediated by the alternative pathway of complement (APC).

Of the few treatment modalities for PNH available, eculizumab is an FDA-approved humanized monoclonal antibody that binds the terminal complement protein C5 and prevents its activation to C5a and C5b, thus inhibiting the formation of the MAC. Eculizumab decreases intravascular hemolysis, as well as reduces the risk for thrombosis. Chronic treatment with eculizumab is generally efficacious in more than 50% of PNH patients requiring therapy, resulting in sustained control of intravascular hemolysis, leading to transfusion independence for complete responders to the therapy.

Eculizumab compensates for the CD59 deficiency on PNH red blood cells, but does not compensate for the CD55 deficiency. Indeed, in patients receiving eculizumab, PNH red blood cells begin to accumulate C3 on their cell surface, which leads to opsonins that are recognized by the reticuloendothelial system resulting in extravascular hemolysis, typically in the liver and spleen. Laboratory evidence of extravascular hemolysis in eculizumab-treated patients includes an elevated reticulocyte count, a normal to mildly elevated LDH, varying degrees of anemia, and in most cases a direct Coombs test that is positive for C3 deposition, but not IgG. Therefore, patients treated with eculizumab may develop extravascular hemolysis that is not present in untreated PNH patients. Additionally, treatment with eculizumab may result in the oposinization of red blood cells.

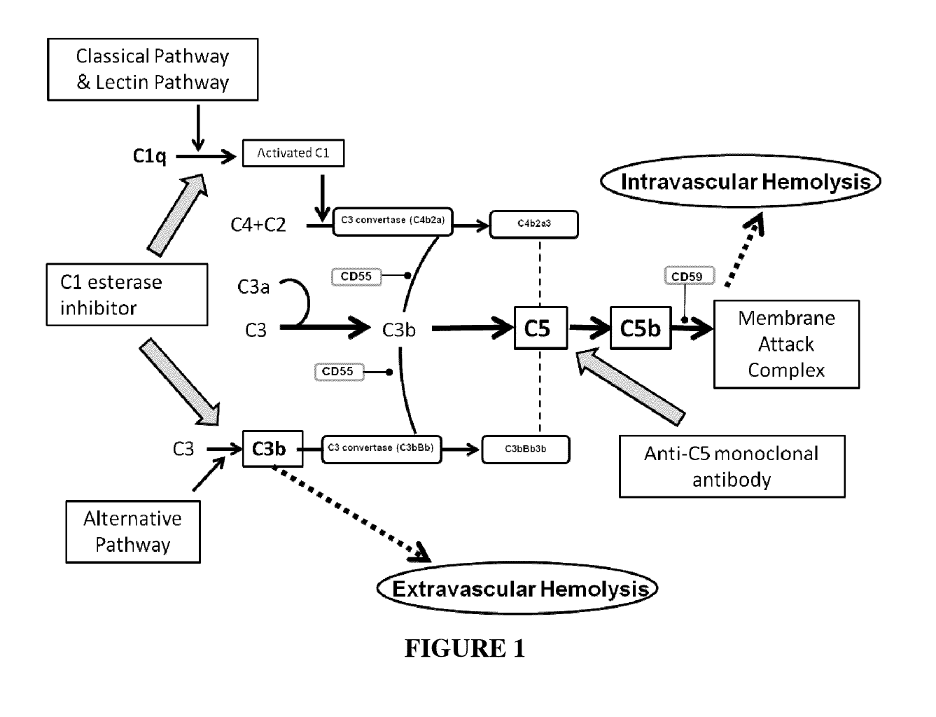

Moreover, C3 accumulation on PNH red blood cells is not observed in untreated patients because complement activation on red blood cells in the absence of eculizumab leads to their rapid elimination due to the MAC. Activation of the complement cascade can be triggered via the APC and when it converges with the classic complement pathway, C3-convertase is formed. C3 is then cleaved into C3a and the opsonizing C3b, which contributes to the formation of C5-convertase. Subsequently, this cleaves C5 into the anaphylatoxin C5a and C5b, thereby initiating formation of the terminal MAC (FIG. 1).

Accordingly, although some patients experiencing PNH, who are taking eculizumab remain asymptomatic, others have poor responses to eculizumab and experience symptomatic persistent anemia. Indeed, extravascular hemolysis may not be present in untreated patients and may be a result of eculizumab therapy, which compensates only for the CD59 deficiency. Thus, there is a need for a treatment regimen involving administration of a complement inhibitor having activity early in the complement cascade, which would not only reduce C3 accumulation on PNH red blood cells, but also inhibit C3 activation by the APC. Such a treatment regimen could be implemented as a monotherapy or in combination with agents such as eculizumab, to effect a complete treatment of disorders such as PNH.

The present invention provides methods of treating or delaying the progression of diseases and disorders that implicate alternative pathway complement immune system activation. Disorders implicating the alternative pathway complement immune system activation include, for example, paroxysmal nocturnal hemoglobinuria (PNH), dense deposit disease (DDD), factor H deficiency, age-related macular degeneration (AMD), atypical hemolytic uremic syndrome (aHUS), and sepsis. In a preferred aspect of the invention, the disorder implicating the alternative pathway complement immune system activation is PNH.

As used herein, the terms “treatment,” “treating,” and the like refer to means for obtaining a desired pharmacologic or physiologic effect, for example. The effect may be prophylactic in terms of completely or partially preventing a condition, appearance, disease, or symptom and/or may be therapeutic in terms of a partial or complete cure for a condition and/or adverse effect attributable to a condition or disease.

The method of the invention further relates to treating or delaying the progression of disorders alleviated by inhibiting alternative pathway complement immune system activation in a patient in need of such treatment, where the method includes administering to the patient a therapeutically effective amount of C1-esterase inhibitor (C1-INH) alone, or in combination with another biologically active agent.

C1 esterase inhibitor (C1-INH) is an endogenous plasma protein in the family of serine protease inhibitors (SERPINs) and has broad inhibitor activity in the complement, contact, and coagulation pathways. C1-INH inhibits the classical pathway of the complement system by binding C1r and C1s and inhibits the mannose-binding lectin-associated serine proteases in the lectin pathway. A nanofiltered plasma derived C1-INH (Cinryze®; Viropharma) is FDA approved for routine prophylaxis against angioedema attacks in adolescent and adult patients with hereditary angioedema (HAE), a disease characterized by constitutional deficiency or dysfunction of endogenous C1 esterase inhibitor.

Cinryze® is known to be well tolerated in humans via the experience in patients with HAE studied in randomized trials as well as in an extension trial. The most frequent adverse events reported at the doses used for HAE were headaches and nasopharyngitis. In more than four years of post-marketing surveillance, there have been no safety concerns for infectious events that could be attributed to Cinryze®. Moreover, plasma derived formulations of C1-INH have been evaluated for their clinical use in pilot studies of sepsis, ischemia-reperfusion injury, and capillary leak in bone marrow transplantation. Thus, C1-INH is an ideal therapeutic, either alone or as part of a combination therapy, for diseases that implicate, for example, the classical complement pathway (e.g., antibody-mediated diseases) and of the lectin pathway (e.g., ischemia reperfusion injury).

Moreover, C1-INH blocks the accumulation of C3 degradation products on CD55 deficient red blood cells and inhibits APC-mediated hemolysis. Thus, C1-INH is an inhibitor of extravascular hemolysis, which may be due to PNH or, for example, incident to the treatment of PNH by eculizumab. With respect to the present invention, the C1-INH may be, for example, an isolated human plasma derived C1-INH (hC1-INH) or a recombinant C1-INH (rC1-INH). In a preferred aspect, the C1-INH is rC1-INH.

The term “effective amount,” as used herein, refers to the quantity of a compound or composition that achieves a beneficial clinical outcome when the compound or composition is administered to a patient. For example, when a composition of the invention is administered to a patient with, for example, PNH, a “beneficial clinical outcome” includes the reduction in intravascular hemolysis, extravascular hemolysis, or both and/or an increase in the longevity of the patient.

The term “isolated,” as used herein in describing a material, for example, refers to material removed from its original environment (e.g., the natural environment if it is naturally occurring). For example, a naturally-occurring polypeptide (i.e., protein) present in a living animal is not isolated, but the same polypeptide, separated from some or all of the coexisting materials in the natural system, is isolated.

Moreover, the “polypeptides” or “proteins” used in practicing the present invention may be natural proteins, synthesized proteins, or may be preferably recombinant proteins. Further, the proteins described herein can be naturally purified products, or chemically synthesized products, or recombinant products from prokaryotic or eukaryotic hosts (e.g., bacteria, yeast, higher plant, insect, or mammalian cell). Such proteins can be glycosylated or non-glycosylated according to the different hosts used.

Turning to the recombinant proteins used in practicing the invention, the recombinant C1-INH (rC1-INH) proteins can be expressed or produced by conventional recombinant DNA technology, using a polynucleotide sequence specific to C1-INH as known in the art. Generally, such recombinant procedure comprises the following steps:
(1) transfecting or transforming the appropriate host cells with the polynucleotide or its variants encoding C1-INH protein of the invention or the vector containing the polynucleotide;
(2) culturing the host cells in an appropriate medium; and
(3) isolating or purifying the protein from the medium or cells.

Regarding the invention more generally, in methods of treating diseases implicating the alternative pathway complement immune system activation and on C3 degradation product accumulation, C1-INH may be used in combination with an additional biologically active agent effective for treating or delaying the progression of a disorder such as, for example, PNH, dense deposit disease (DDD), factor H deficiency, age-related macular degeneration (AMD), atypical hemolytic uremic syndrome (aHUS), and sepsis. In preferred aspects of the invention, the additional biologically active agent is effective for treating or delaying the progression of PNH. Moreover, such biologically active agents may not provide complete treatments for each of the above-referenced disorders and may in fact provide merely a partial or incomplete treatment, such as in the case of eculizumab. Additionally, TT30 is a recombinant human fusion protein and may prevent opsonization and extravascular hemolysis. Accordingly, the biologically active agent is preferably eculizumab, TT30, or a combination thereof. However, in a particularly preferred aspect, the biologically active agent is eculizumab. Therefore, in certain preferred aspects of the method of the invention, a C1-INH may be administered to a patient in combination with eculizumab (e.g., co-administration).

When applying the method of the invention by co-administration, where separate dosage formulations are used, the C1-INH and biologically active agent can be administered concurrently, or separately at staggered times, i.e., sequentially. In practice, the agents of the invention may be administered as separate dosage units or formulated for administration together, according to procedures well known to those skilled in the art. See, for example, Remington: The Science and Practice of Pharmacy, 20^{th} ed., A. Genaro et al., Lippencot, Williams & Wilkins, Baltimore, Md. (2000). Preferably, the C1-INH is administered concurrently with the biologically active agent. In other preferred co-administration strategies, the C1-INH may be administered, for example, before administration of the biologically active agent, after administration of the biologically active agent, or concomitantly with the administration of the biologically active agent. Additionally, the C1-INH may be administered concurrently with the biologically active agent where the amount or concentration of the biologically active agent is decreased or tapered with respect to the C1-INH, wherein the amount or concentration of the C1-INH is increased, decreased, or fixed.

Suitable methods of introduction of compositions of the invention to a patient include but are not limited to intradermal, intramuscular, intraperitoneal, intravenous, subcutaneous, intranasal, intraocular, epidural, and oral routes. Moreover, compositions of the invention may be administered by infusion or bolus injection, by absorption through epithelial or mucocutaneous linings (e.g., oral mucosa, rectal, and intestinal mucosa, etc.). Administration may further be systemic or local. And administration can be acute or chronic (e.g., daily, weekly, monthly, etc.).

The orally administered dosage unit may be in the form of tablets, caplets, dragees, pills, semisolids, soft or hard gelatin capsules, aqueous or oily solutions, emulsions, suspensions or syrups. Representative examples of dosage forms for parenteral administration include injectable solutions or suspensions, suppositories, powder formulations, such as microcrystals or aerosol spray. The composition may also be incorporated into a conventional transdermal delivery system.

Additionally, in certain situations, compounds used in practicing the invention may be delivered as pharmaceutical compositions that include a pharmaceutically acceptable carrier medium. For example, the invention includes a pharmaceutical composition for treating or delaying the progression of a disorder alleviated by inhibiting alternative pathway complement immune system activation in a patient in need of such treatment, the composition comprising a C1-esterase inhibitor (C1-INH); an additional biologically active agent, such as eculizumab, TT30, or a combination thereof; and a pharmaceutically acceptable carrier medium. As used herein, the expression “pharmaceutically acceptable carrier medium” includes any and all solvents, diluents, or other liquid vehicle, dispersion or suspension aids, surface agent agents, isotonic agents, thickening or emulsifying agents, preservatives, solid binders, lubricants, fillers and the like as suited for the particular dosage form desired. Remington: The Science and Practice of Pharmacy, 20th edition, A. R. Genaro et al., Part 5, Pharmaceutical Manufacturing, pp. 669-1015 (Lippincott Williams & Wilkins, Baltimore, Md./Philadelphia, Pa.) (2000)) discloses various carriers used in formulating pharmaceutical compositions and known techniques for the preparation thereof. Except insofar as any conventional pharmaceutical carrier medium is incompatible with the compositions described herein, such as by producing an undesirable biological effect or otherwise interacting in an deleterious manner with any other component(s) of a formulation comprising the active agent(s), its use is contemplated to be within the scope of this invention.

More specifically, in the production of solid dosage forms the pharmaceutical composition may be mixed with pharmaceutically inert, inorganic or organic excipients, such as lactose, sucrose, glucose, gelatine, malt, silica gel, starch or derivatives thereof, talc, stearic acid or its salts, dried skim milk, vegetable, petroleum, animal or synthetic oils, wax, fat, polyols, and the like. Liquid solutions, emulsions or suspensions or syrups one may use excipients such as water, alcohols, aqueous saline, aqueous dextrose, polyols, glycerine, lipids, phospholipids, cyclodextrins, vegetable, petroleum, animal or synthetic oils. Suppositories may include excipients, such as vegetable, petroleum, animal or synthetic oils, wax, fat and polyols. Aerosol formulations may include compressed gases suitable for this purpose, such as oxygen, nitrogen and carbon dioxide. The pharmaceutical composition or formulation may also contain one or more additives including, without limitation, preservatives, stabilizers, e.g., UV stabilizers, emulsifiers, sweeteners, salts to adjust the osmotic pressure, buffers, coating materials and antioxidants.

The present invention further provides controlled-release, sustained-release, or extended-release therapeutic dosage forms for the pharmaceutical composition, in which the composition is incorporated into a delivery system. This dosage form controls release of the active agent(s) in such a manner that an effective concentration of the active agent(s) in the bloodstream can be maintained over an extended period of time, with the concentration in the blood remaining relatively constant, to improve therapeutic results and/or minimize side effects. Additionally, a controlled-release system would provide minimum peak to trough fluctuations in blood plasma levels of the active agent.

Additionally, various delivery systems are known and can be used to administer compositions that comprise C1-INH, or C1-INH in combination with a biologically active agent, such as eculizumab. For example, such compositions may be encapsulated in liposomes, microparticles, and microcapsules, for example.

The methods of the present invention will normally include medical follow-up to determine the therapeutic or prophylactic effect brought about in the patient undergoing treatment with the compound(s) and/or composition(s) described herein.

Turning to the use of C1-INH as a treatment for disease more specifically, C1-INH may be used for preventing hemolysis in PNH red blood cells ex vivo.

For certain patients, eculizumab is highly efficacious and has been a life-saving PNH treatment. However, up to 20% of patients remain transfusion dependent due to chronic persistent extravascular hemolysis. Extravascular hemolysis in PNH patients receiving eculizumab results from CD55 deficiency. Activation of C3 to C3b on the surface of PNH red blood cells by the APC C3 convertase results in degradation products of C3, which act as opsonins that mediate extravascular hemolysis as a consequence of interaction with complement receptor-expressing cells (macrophages and B lymphocytes) that are present in the liver and spleen. However, it has been demonstrated, in accordance with this invention, that plasma derived C1-INH (Cinryze®) prevents lysis, induced by the alternative complement pathway, of PNH red blood cells in human serum Importantly, C1-INH is able to block the accumulation of C3 degradation products on CD55 deficient red blood cells from PNH patients who are being treated with eculizumab. These results tend to suggest that patients with PNH could benefit from blocking the APC activation in order to inhibit both C3 deposition and hemolysis (FIG. 1). Patients who do not respond to eculizumab may respond to a C1-INH, either alone or in combination with C5 blockade (e.g., eculizumab).

In other studies, patients with PNH have shown that there are additional strategies of complement inhibition that could further improve on the already demonstrated efficacy of eculizumab. These studies were performed to determine the effects of C1 inhibition on hemolysis and C3 deposition when the complement is activated via the APC. The results showed that, the unadulterated commercial form of plasma-derived C1-INH did prevent C3 deposition and APC hemolysis of PNH cells. Preclinical models of an anti-C3 monoclonal antibody have been shown to affect the activity of the C3/C5 convertases, thus preserving the classical pathway and effectively inhibiting the hemolysis of the PNH erythrocytes in vitro.

The present invention demonstrates that the drug Cinryze®, currently approved by the FDA and EMA for use in HAE patients, inhibits C3 deposition and APC activation.

Additionally, the present invention provides an analysis on the sera of patients who already have inhibition of complement via the MAC through dosing of eculizumab. The data disclosed herein suggests that C1-INH is effective to block the APC, as is consistent with its mechanism of action.

In certain embodiments of the invention, the inhibition of hemolysis did not appreciably occur until 3 units per mL were administered to plated cells. Physiologic C1-INH is approximately 1 unit per mL of human serum.

The results of the experiments described in the following examples demonstrate that commercially available plasma-derived C1-INH can inhibit both APC-mediated hemolysis and C3 deposition. Thus, C1-INH provides for a treatment regimen that inhibits both intra- and extra-vascular hemolysis. Accordingly, there is a role for inhibition of earlier phases of the complement cascade than those currently inhibited by eculizumab, in cases of incomplete responders or non-responders to eculizumab therapy.

The following examples are provided to describe the invention in further detail. These examples are provided for illustrative purposes only and are not intended to limit the invention in any way.

### EXAMPLE

The commercial product Cinryze®, unadulterated, inhibits C3 deposition and the APC on PNH red blood cells in patients treated with eculizumab.

Here, peripheral blood of healthy donors and PNH patients treated with eculizumab were obtained in EDTA tubes according to protocols approved by the institutional review board at Johns Hopkins University. Commercial vials of Cinryze® were used for C1 inhibition assays ex vivo. Serial dilutions were prepared for a dose response. Hemolysis experiments for erythrocytes from PNH patients and healthy donor and dose response curves performed. Flow cytometry was used to analyze deposition of C3 activation fragments on intact and lysed PNH erythrocytes (ghosts).

As demonstrated below, plasma derived C1-INH (Cinryze®) prevents lysis induced by the alternative complement pathway of PNH red blood cells in human serum Importantly, C1-INH blocks the accumulation of C3 degradation products on CD55 deficient red blood cells from PNH patients treated with eculizumab.

### MATERIALS AND METHODS

Blood Samples. Peripheral blood of healthy donors and PNH patients treated with eculizumab were obtained in EDTA tubes according to protocols approved by the institutional review board at Johns Hopkins University after written informed consent. PNH type III erythrocytes were defined as the percentage of CD55 deficient erythrocytes in whole blood and measured by flow cytometry with previously described methods known in the art. (Brodsky, Mukhina et al. 2000; Craig et al. 2010). Patients were chosen who were aged 18 years or older, with a PNH type III erythrocyte proportion >5% while receiving eculizumab. Clinical parameters for their ongoing hemolysis were noted at the time of the sampling.

C1 Esterase Inhibitor. Commercial vials of Cinryze® were used for C1 inhibition assays ex vivo. Serial dilutions were prepared for dose response.

Hemolysis experiments for erythrocytes from PNH patients and healthy donors. Erythrocytes were centrifuged, the buffy coat was aspirated, and the cells were thoroughly washed in gelatine veronal buffer (GVB) before each experiment (Wilcox, Ezzell et al. 1991). Tests for the susceptibility of erythrocytes to APC-mediate lysis followed previously described methods. (Wilcox, Ezzell et al. 1991). Briefly, erythrocytes were washed with the GVB saline, pH 7.4, and incubated at final hematocrit of 2% with 1:2 diluted human serum, type AB in the GVB⁺² saline, pH 6.4 at 37° C. To estimate dose response ex vivo, incremental concentrations of the plasma derived C1-INH of 0 U/mL, 3 U/mL, 6 U/mL, 9 U/mL, and 12 U/mL were mixed with serum before adding erythrocytes. After 1 hour, the erythrocytes were pelleted by centrifugation at 13,000 rpm for 5 minutes, and the optical density at 415 nm of an aliquot of the recovered supernatant was used to calculate the percentage lysis. The mean absorbance values with standard deviations from three independent experiments are presented. Healthy donor samples reconstructed in acidified serum-EDTA were processed similarly and used to illustrate background non-complement mediated lysis.

C3 Inhibition. Flow cytometry was used to analyze deposition of C3 activation fragments on intact and lysed PNH erythrocytes (ghosts). After incubation, the ghosts and intact erythrocytes were collected by centrifugation at 13,000 rpm for 5 minutes, washed three times with the ice-cold phosphate buffered saline, pH 7.4, and subjected to staining with FITC-conjugated anti-C3/C3b fragment antibody and PE-conjugated anti-CD55 antibody, followed by flow cytometric analysis.

### RESULTS

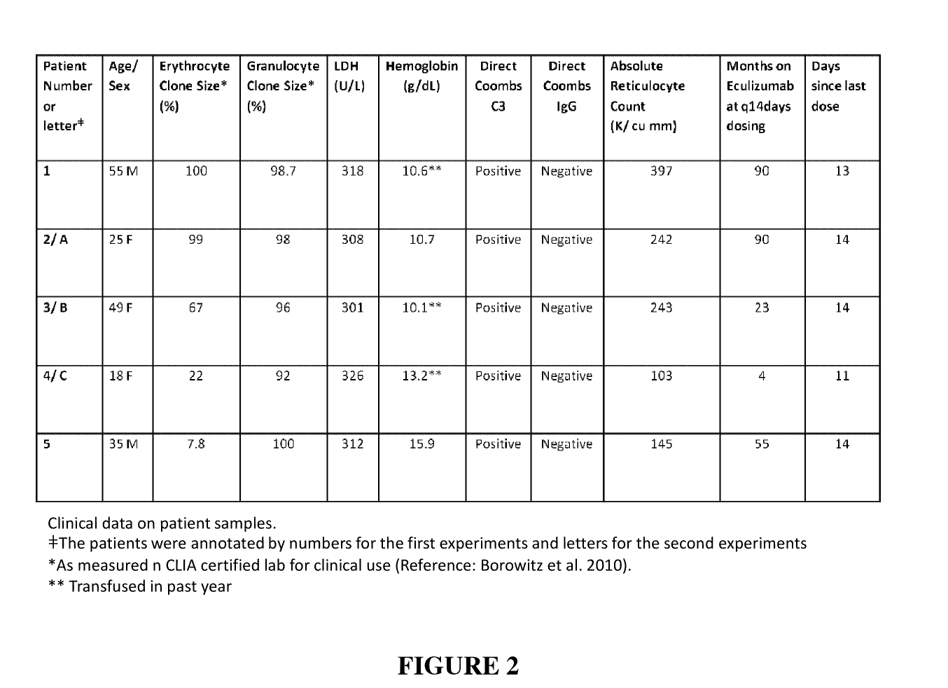

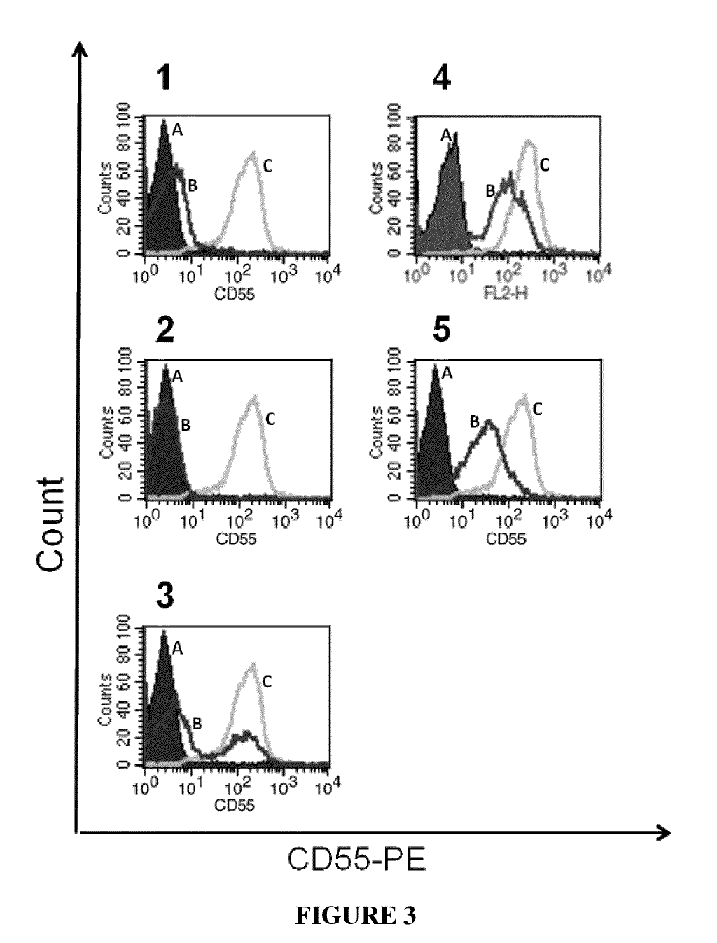

Patients receiving eculizumab have persistent extravascular hemolysis. The clinical information for each patient on the day their serum sample was obtained is provided in FIG. 2. These samples were drawn prior to eculizumab dosing if it was due on that day. These baseline data are presented to show persistent extravascular hemolysis despite long term and ongoing inactivation of complement with eculizumab at standard dosing. FIG. 3 shows red blood cell CD55 expression for each patient the day the assay was performed. These are comparable to the clone sizes performed for clinical use within 3 months of these assays as shown in FIG. 2. No patient received a red blood cell transfusion within 120 days of the assay.

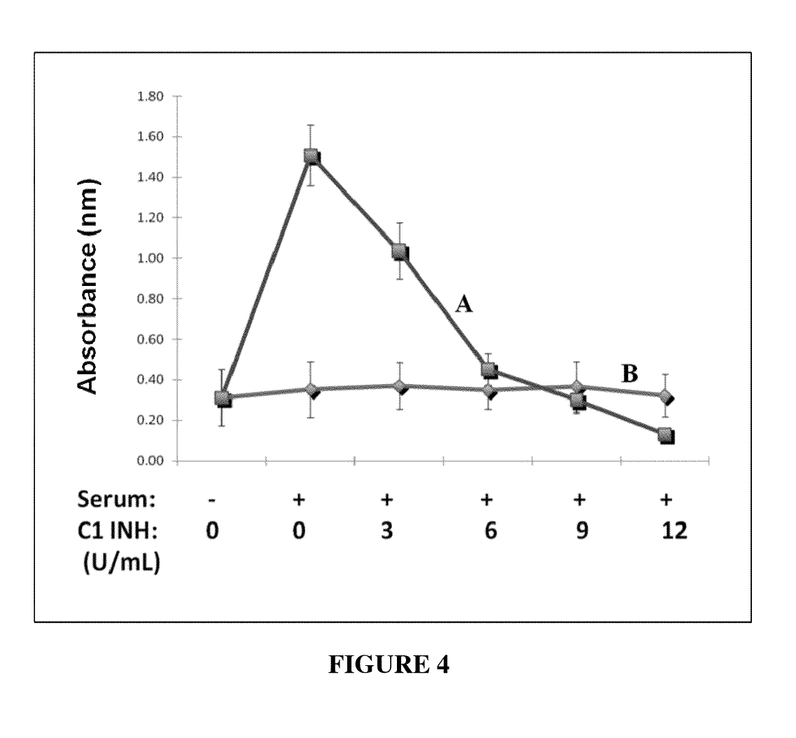

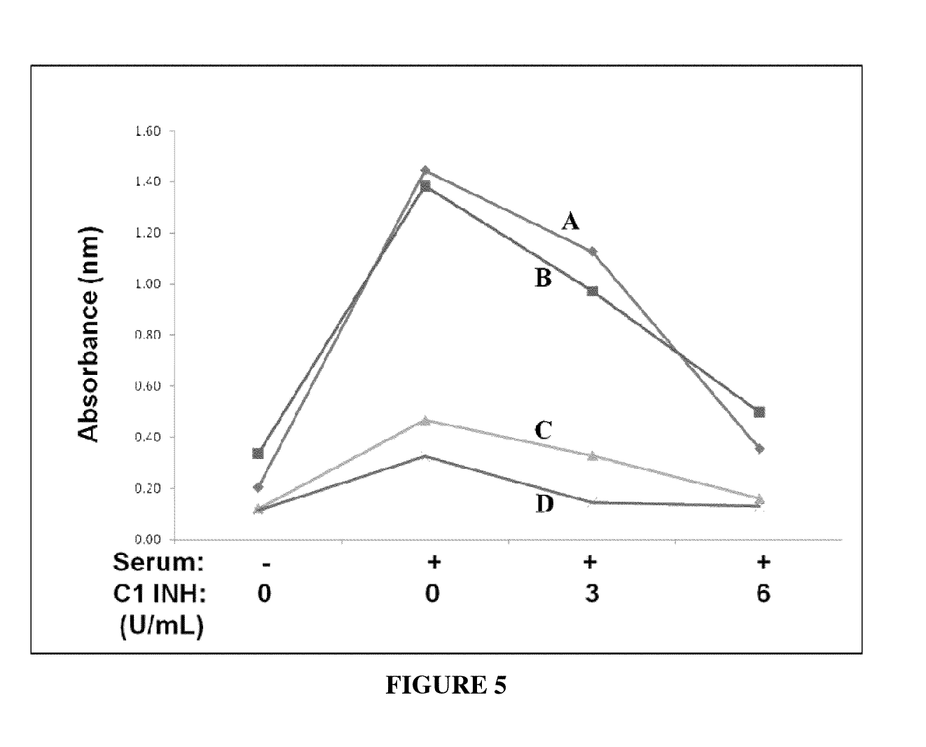

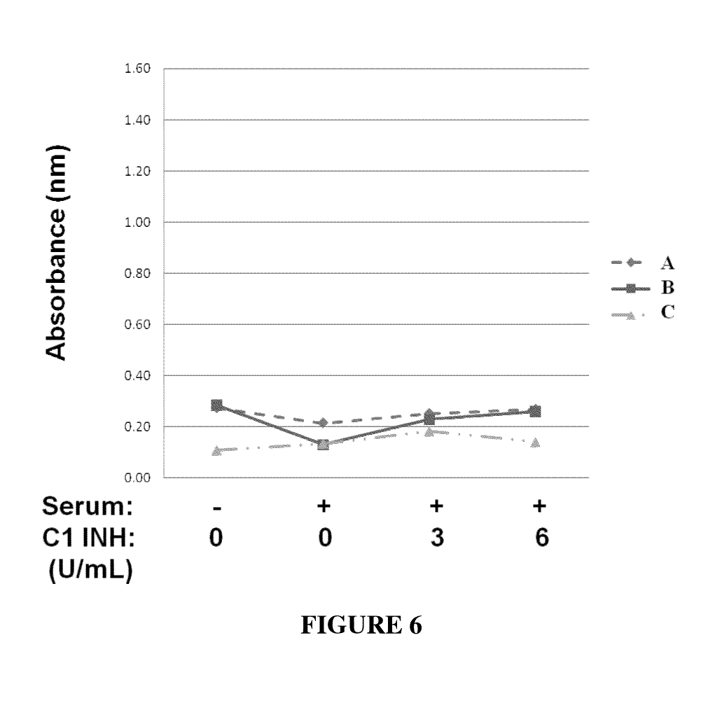

C1 inhibition is concentration dependent. To investigate if C1-INH inhibits complement mediated hemolysis of PNH erythrocytes in a concentration dependent manner, the serum of patient 2 was incubated for 1 hour at 37° C. with acidified normal serum and acidified heat-inactivated serum containing with increasing concentration of C1-INH as above. After incubation, hemolysis was measured using the concentration of supernatant hemoglobin (determined by spectrophotometry at 415 nm). To further determine if erythrocyte clone size affected the amount of lysis, the sera of patients 1-4 were prepared as above and then incubated with C1-INH in FIG. 4. FIG. 5 demonstrates that hemolysis of PNH red blood cells in acidified serum correlates with the percentage of CD55 deficient red blood cells. Moreover, C1-INH attenuates hemolysis in all PNH patient red blood cells studied. Red blood cells from healthy controls did not hemolyze in response to acidified serum (FIG. 6).

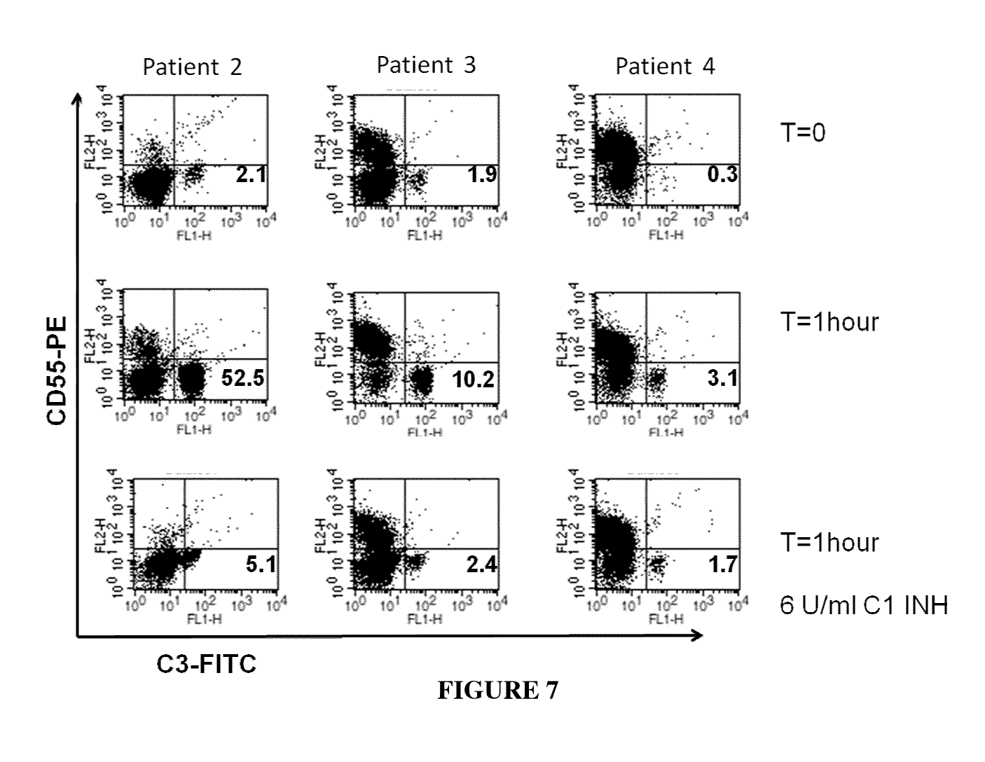

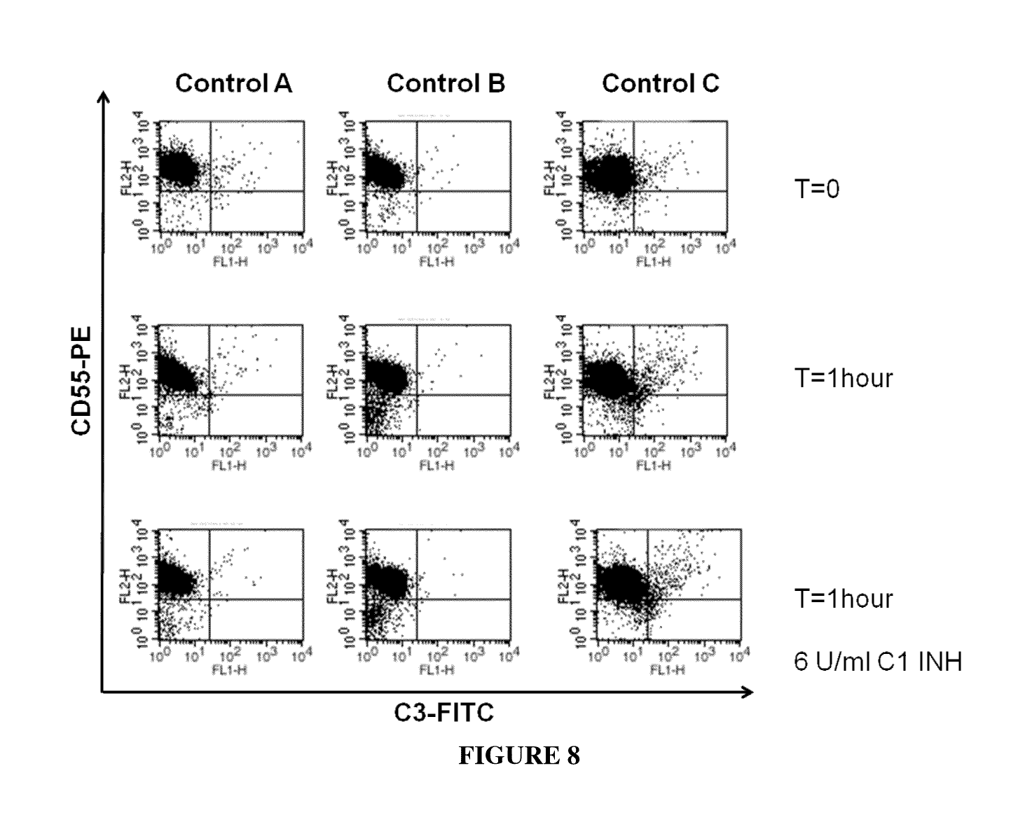

C1 inhibition blocks APC-medicated deposition of C3. It has been demonstrated that patients with PNH treated with eculizumab develop the extravascular hemolysis because the drug blocks MAC-induced lysis, thus allowing the CD55/CD59-deficient cells to become opsonised with activation and degradation products of C3 as a consequence of unrestricted activation of the APC. To investigate whether C1-INH can protect cells from C3 deposition, we exposed PNH red blood cells to acidified serum and assayed for deposition of C3 fragments before and after increasing concentrations of C1-INH. All PNH patients were actively being treated with eculizumab. At time 0, small amounts of C3 deposition was observed on the PNH red blood cells (CD55 deficient), but not the normal red blood cells (CD55 positive) from all three patients studied. The amount of C3 deposition was increased in all 3 subjects after incubation in acidified serum. However, co-incubation of C1-INH in the acidified serum markedly attenuated the amount of C3 deposition on the CD55 deficient red blood cells (FIG. 7). FIG. 8 illustrates that this is not present in healthy donors.

The above-described results demonstrate that plasma-derived C1-INH can inhibit both APC-mediated hemolysis and C3 deposition. The PNH patients in this example receiving eculizumab demonstrated persistent extravascular hemolysis. It is shown herein that C1 inhibition is concentration dependent (FIG. 4). Furthermore, C1-INH attenuates hemolysis in all PNH patient red blood cells studied. Red blood cells from healthy controls did not hemolyze in response to acidified serum. Finally, using flow cytometry it is demonstrated that C1 inhibition blocks APC-medicated deposition of C3.

Thus, C1-INH inhibits earlier phases of the complement cascade than that currently inhibited by eculizumab for incomplete for non-responders to that therapy. Therefore, C1-INH may be provided as a treatment of diseases that implicate APC-mediated hemolysis and C3 deposition, such as PNH. Moreover, it is evident that C1-INH may be presented in combination with eculizumab to provide efficacious and complete treatments to patients suffering from diseases such as PNH.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions and methods described herein that embody the present invention can, in alternate embodiments, be more specifically defined by any of the transitional terms “comprising,” “consisting essentially of,” and “consisting of.”

### REFERENCES

Borowitz, M. J., F. E. Craig, et al. (2010). “Guidelines for the diagnosis and monitoring of paroxysmal nocturnal hemoglobinuria and related disorders by flow cytometry.” Cytometry B Clin. Cytom. 78(4): 211-230.
Brodsky, R. A., G. L. Mukhina, et al. (2000). “Improved detection and characterization of paroxysmal nocturnal hemoglobinuria using fluorescent aerolysin.” Am. J. Clin. Pathol. 114(3): 459-466.
DeZern A E, Don D, Brodsky R A. (2013). “Predictors of hemoglobin response to eclulizumab therapy in paroxysmal noctornual hemoglobinuria.” Eur. J. Haematol. 90(1): 16-24.
Wilcox, L. A., J. L. Ezzell, et al. (1991). “Molecular basis of the enhanced susceptibility of the erythrocytes of paroxysmal nocturnal hemoglobinuria to hemolysis in acidified serum.” Blood 78(3): 820-829.

What is claimed is:

1. A method of treating or delaying the progression of a disorder alleviated by inhibiting alternative pathway complement immune system activation in a patient in need of such treatment, the method comprising administering a therapeutically effective amount of C1-esterase inhibitor (C1-INH).

2. A method of treating or delaying the progression of a disorder alleviated by inhibiting the accumulation of protein degradation products of a protein on red blood cells in a patient in need of such treatment, the method comprising administering a therapeutically effective amount of C1-esterase inhibitor (C1-INH).

3. The method according to claim 2, wherein the protein is C3.

4. The method according to claim 2, wherein the C1esterase inhibitor (C1INH) comprises a human plasma-derived C1-INH (hC1-INH) or a recombinant C1-INH (rC1-INH).

5. The method according to claim 2, wherein the disorder is selected from the group consisting of paroxysmal nocturnal hemoglobinuria (PNH), dense deposit disease (DDD), factor H deficiency, age-related macular degeneration (AMD), atypical hemolytic uremic syndrome (aHUS), and sepsis.

6. The method according to claim 2, comprising administering an additional biologically active agent effective for treating or delaying the progression of a disorder selected from the group consisting of paroxysmal nocturnal hemoglobinuria (PNH), dense deposit disease (DDD), factor H deficiency, age-related macular degeneration (AMD), atypical hemolytic uremic syndrome (aHUS), and sepsis.

7. The method according to claim 6, comprising administering eculizumab, TT30, or a combination thereof, as the additional biologically active agent.

8. The method according to claim 6, wherein the C1-INH and the biologically active agent are administered concurrently.

9. The method according to claim 6, wherein the C1-INH and the biologically active agent are administered sequentially.

10. A method of treating or delaying the progression of paroxysmal nocturnal hemoglobinuria (PNH) in a patient in need of such treatment, the method comprising administering therapeutically effective amounts of at least a C1-esterase inhibitor (C1-INH) and eculizumab.

11. The method according to claim 10, wherein the C1-esterase inhibitor (C1-INH) comprises a human plasma derived C1-INH (hC1INH) or a recombinant C1-INH (rC1INH).

12. The method according to claim 10, wherein the C1-INH and eculizumab are administered concurrently.

13. The method according to claim 10, wherein the C1-INH and eculizumab are administered sequentially.

14. A method of treating the opsonization of blood cells in an organ in a patient in need of such treatment, the method comprising administering a therapeutically effective amount of a C1-esterase inhibitor (C1-INH).

15. The method of claim 14, wherein the C1-esterase inhibitor (C1-INH) comprises a human plasma derived C1-INH (hC1-INH) or a recombinant C1-INH (rC1-INH).

16. The method of claim 14, wherein the organ is selected from the group consisting of spleen, liver, and a combination thereof.

17. The method according to claim 14, wherein the opsonization of blood cells is due to eculizumab treatment.

18. A pharmaceutical composition for treating or delaying the progression of a disorder alleviated by inhibiting alternative pathway complement immune system activation in a patient in need of such treatment, the composition comprising
a C1-esterase inhibitor (C1-INH);
a biologically active agent selected from the group consisting of eculizumab, TT30, or a combination thereof; and
a pharmaceutically acceptable carrier medium.

19. The method according to claim 1, wherein the C1-esterase inhibitor (C1-INH) comprises a human plasma-derived C1-INH (hC1-INH) or a recombinant C1-INH (rC1-INH).

20. The method according to claim 1, wherein the disorder is selected from the group consisting of paroxysmal nocturnal hemoglobinuria (PNH), dense deposit disease (DDD), factor H deficiency, age-related macular degeneration (AMD), atypical hemolytic uremic syndrome (aHUS), and sepsis.

21. The method according to claim 1, comprising administering an additional biologically active agent effective for treating or delaying the progression of a disorder selected from the group consisting of paroxysmal nocturnal hemoglobinuria (PNH), dense deposit disease (DDD), factor H deficiency, age-related macular degeneration (AMD), atypical hemolytic uremic syndrome (aHUS), and sepsis.
